(19) United States
(12) Patent Application Publication
(10) Pub. No.: US 2024/0078626 A1
(43) Pub. Date: Mar. 7, 2024

(54) PERSON DETERMINATION APPARATUS, PERSON DETERMINATION METHOD, AND NON-TRANSITORY STORAGE MEDIUM

(71) Applicant: NEC Corporation, Tokyo (JP)

(72) Inventor: Kazuya Kawakami, Tokyo (JP)

(73) Assignee: NEC Corporation, Tokyo (JP)

(21) Appl. No.: 18/238,121

(22) Filed: Aug. 25, 2023

(30) Foreign Application Priority Data
Sep. 1, 2022 (JP) 2022-139016

(51) Int. Cl. G06Q 50/26; G16H 10/60

(52) U.S. Cl. CPC G06Q 50/265; G16H 10/60

(57) ABSTRACT

A person determination apparatus includes a selection unit and an information generation unit. The selection unit uses biometric information of a search target person, and thereby selects a candidate having a possibility of being the search target person. The information generation unit uses medical information concerning a medical history of the search target person and the medical information of the candidate, and thereby generates possibility information being information concerning a possibility that the candidate is the search target person.

This application is based upon and claims the benefit of priority from Japanese patent application No. 2022-139016, filed on Sep. 1, 2022, the disclosure of which is incorporated herein in its entirety by reference.

### BACKGROUND

### Technical Field

The present invention relates to a person determination apparatus, a person determination method, and a program.

### Background Art

In recent years, there are more occasions where biometric information of a person is used at a time of determining the person himself or herself. For example, in an image processing system described in PTL 1 (Japanese Patent Application Publication No. 2017-216005), an image capturing apparatus acquires an authentication method decided based on information concerning an authentication target person. An image capturing person uses an image capturing apparatus, and thereby captures an image of the authentication target person being an image capture target as a subject. Then, an authentication unit of the image capturing apparatus performs authentication processing for the authentication target person, based on the acquired authentication method. This authentication processing is performed based on at least one of biometric information and personal information of a patient.

Further, PTL 2 (Japanese Patent Application Publication No. 2018-169653) describes an authentication apparatus that performs the following processing. First, the authentication apparatus stores, in association with one another, combination information of types selected from each of a plurality of categories, user identification information, and biometric authentication information of a user. Then, the authentication apparatus displays images of a plurality of the types for each category. A user selects, on this screen, the type for each category, and inputs combination information of a plurality of these types. The authentication apparatus acquires user information associated with the input combination information of a plurality of the types, compares the biometric information associated with the user information, with biometric authentication information input to the authentication apparatus, and decides as authentication success when both match with each other.

Further, PTL 3 (Japanese Patent Application Publication No. 2019-155610) describes a print system having the following function. In a case of printing print data stored in a security server whose security level is high, the print system performs user authentication by two-factor authentication of IC card authentication and biometric authentication. On the other hand, in a case of printing print data stored in a server other than the security server, the print system performs user authentication by one-factor authentication of IC card authentication.

### SUMMARY

There is a possibility that biometric information changes with aging. Thus, a problem exists in an arising possibility that, when biometric information serving as a master is old, accuracy at a time of determining a target person by using the biometric information declines. This problem can occur in any of above-described documents.

In view of the above-described problem, one example of an object of the present invention is to provide a person determination apparatus, a person determination method, and a program that are capable of determining a person with high accuracy.

According to one aspect of the present invention, there is provided a person determination apparatus including:
at least one memory configured to store instructions; and
at least one processor configured to execute the instructions to perform operations including:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

According to one aspect of the present invention, there is provided a person determination method including,
by a computer:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

According to one aspect of the present invention, there is provided a non-transitory storage medium storing a program causing a computer to execute a person determination method, the person determination method including:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

According to one aspect of the present invention, a person determination apparatus, a person determination method, and a program that are capable of determining a person with high accuracy can be acquired.

### BRIEF DESCRIPTION OF THE DRAWINGS

The above and other objects, advantages and features of the present invention will be more apparent from the following description of certain preferred example embodiments taken in conjunction with the accompanying drawings, in which:

FIG. 1 is a diagram illustrating an outline of a person determination apparatus according to an example embodiment.

FIG. 2 is a diagram for illustrating one example of a use environment of the person determination apparatus.

FIG. 3 is a diagram illustrating one example of a functional configuration of the person determination apparatus.

FIG. 4 is a diagram illustrating one example of information stored by a search target person storage unit.

FIG. 5 is a diagram illustrating one example of information stored by a candidate storage unit.

FIG. 6 is a diagram illustrating a hardware configuration example of the person determination apparatus.

FIG. 7 is a flowchart illustrating a first example of processing performed by the person determination apparatus.

FIG. 8 is a flowchart illustrating an altered example of FIG. 7.

### EXAMPLE EMBODIMENT

The invention will be now described herein with reference to illustrative example embodiments. Those skilled in the art will recognize that many alternative example embodiments can be accomplished using the teachings of the present invention and that the invention is not limited to the example embodiments illustrated for explanatory purposes.

Hereinafter, an example embodiment of the present invention is described below with reference to the drawings. Note that, in all the drawings, the similar constituent elements are denoted by the similar reference signs, and the description thereof is appropriately omitted.

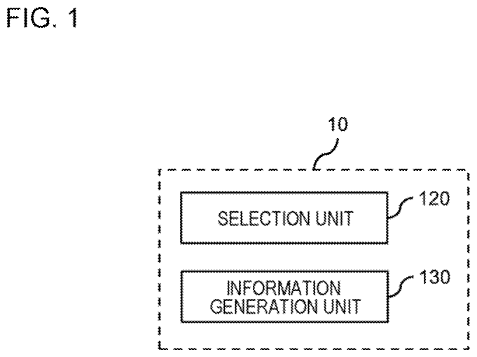

FIG. 1 is a diagram illustrating an outline of a person determination apparatus 10 according to an example embodiment. The person determination apparatus 10 includes a selection unit 120 and an information generation unit 130. The selection unit 120 uses biometric information of a search target person, and thereby selects a candidate having a possibility of being the search target person. The information generation unit 130 uses medical information concerning a medical history of the search target person and the medical information of the candidate, and thereby generates possibility information as information concerning a possibility that the candidate is the search target person.

According to the person determination apparatus 10, the possibility information is generated by using the medical information concerning the medical history of the search target person and the medical information of the candidate. Accordingly, the search target person can be determined from among candidates, with high accuracy.

One example of the search target person is a missing person. In this case, the candidate is selected from unidentified persons. Conversely, there is a case where the search target person is an unidentified person. In this case, the candidate is selected from among missing persons. In these cases, when a certain length of years have passed since the missing person went missing, there is a possibility that biometric information, especially face information, of the missing person has changed. On the other hand, current biometric information is used as biometric information of the unidentified person. Thus, a difference occurs in generation time of the two pieces of the biometric information, and resulting from this, there is an arising possibility that the search target person cannot be determined with high accuracy by only the biometric information. Even in such a case, the person determination apparatus 10 can determine a search target person from among candidates, with high accuracy, because of using medical information in addition to biometric information.

Hereinafter, a detailed example of the person determination apparatus 10 is described.

In the following description, one example of biometric information is at least one of the face information, iris information, fingerprint information, and vein information, but is not limited to these. Note that, the face information is at least one of an image of a face and a feature value of the face.

One example of the medical information is at least one item of skeleton information, information concerning a chronic disease, information concerning a blood test, information concerning treatment received in the past, and information concerning a trace left in the body due to past treatment, but is not limited to these items. For example, the medical information may include at least one of an X-ray image, an MRI image, a medical history, a surgical history, a hospital where treatment was received, a doctor who performed treatment, and time when treatment was received. The medical information may include information that concerns a nutritional state and whose example is information indicating dietary contents.

Herein, the skeleton information includes, for example, at least one of: lengths of a plurality of bones constituting a skeleton of a person; and a ratio of lengths of a plurality of specific bones. The information concerning a blood test includes a blood type, for example.

The information concerning treatment received in the past includes information that indicates what kind of treatment was received in the past and whose example is information described in a medical chart. The information concerning a trace left in the body due to past treatment includes, for example, at least one of a surgery trace, a dental treatment trace, and an injury trace. The medical information preferably includes a plurality of the above-described items.

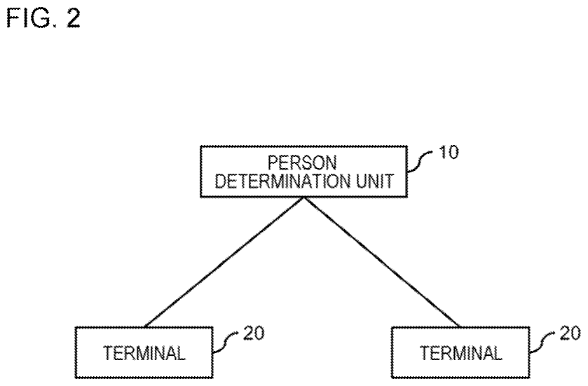

FIG. 2 is a diagram for illustrating one example of a use environment of the person determination apparatus 10. In the example illustrated in the present drawing, the terminal 20 is operated by a person who desires to determine a search target person from among candidates. Hereinafter, this person is referred to as a user. The user inputs, to the terminal 20, various pieces of information concerning the search target person. The input information includes, for example, biometric information and medical information of the search target person. The terminal 20 transmits the input information to the person determination apparatus 10.

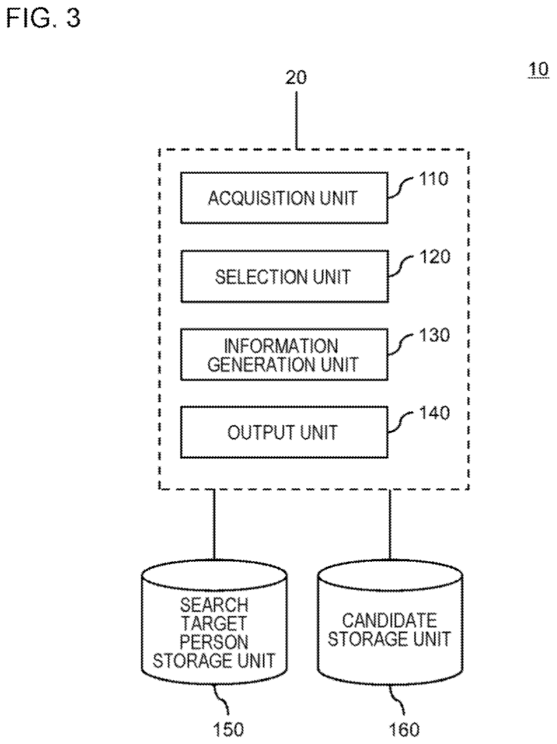

On the other hand, the person determination apparatus 10 can use a storage unit that stores various pieces of information concerning a candidate and whose example is the candidate storage unit 160 described below with reference to FIG. 3. The information stored in this storage unit includes biometric information, medical information, a name, an address, and the like of the candidate. Then, the person determination apparatus 10 uses the biometric information and the medical information of the search target person and the biometric information and the medical information of the candidate, thereby generates possibility information, i.e., information concerning a possibility of being the search target person, and transmits the generated possibility information to terminal 20.

When a plurality of the candidates exist, the possibility information includes, for example, information indicating the person who is among the candidates and who has the highest possibility of being the search target person. This information includes, for example, the name and the address of the person, but may also include other information. The possibility information may also include information of a predetermined number (e.g., three) of the highest ranked candidates when the candidates are arranged in the descending order of possibilities of being the search target person. In each case, the possibility information may include a numerical value indicating a height of possibility that the candidate is the search target person.

When the number of the candidates is one, the possibility information includes, for example, a numerical value indicating a height of possibility that the candidate is the search target person.

Then, the terminal 20 outputs the possibility information acquired from the person determination apparatus 10, and allows the user to recognize the possibility information. One example of this output is to display the possibility information on a display, but there is no limitation to this.

Note that, when a new candidate such as an unidentified person is found, the user inputs various pieces of information concerning this candidate to the terminal 20. The terminal 20 transmits these pieces of information to the person determination apparatus 10. The person determination apparatus 10 stores these pieces of information in the above-described storage unit.

In addition, the person determination apparatus 10 stores the information of the search target person acquired from the terminal 20, in a storage unit, for example, the search target person storage unit 150 described below with reference to FIG. 3.

FIG. 3 is a diagram illustrating one example of a functional configuration of the person determination apparatus 10. The person determination apparatus 10 includes an acquisition unit 110, a selection unit 120, an information generation unit 130, and an output unit 140, and can use the search target person storage unit 150 and the candidate storage unit 160.

The search target person storage unit 150 stores various pieces of information concerning the search target person. The candidate storage unit 160 stores various pieces of information concerning the candidate. In the example illustrated in the present drawing, the search target person storage unit 150 and the candidate storage unit 160 are parts of the person determination apparatus 10. However, the search target person storage unit 150 and the candidate storage unit 160 may be located outside the person determination apparatus 10.

The acquisition unit 110 acquires information transmitted from terminal 20. This information is, for example, various pieces of information concerning the candidate in one case, or is various pieces of information concerning the search target person in another case. Examples of “various pieces of information” are a name, an address, an age, biometric information, and medical information. For example, in an assumed case, one of the candidate and the search target person is a missing person, and the other of the candidate and the search target person is an unidentified person. In this case, the information of the missing person includes a name, an address at the time of going missing, an age at present or at the time of going missing, biometric information at the time of going missing, and medical information at the time of going missing. The information of the unidentified person includes a name (a pseudonym in some cases) at the time of being found, an address at the time of being found, an estimated age at the time of being found, biometric information at the time of being found, and medical information at the time of being found. These pieces of information are stored in the search target person storage unit 150 and the candidate storage unit 160.

The selection unit 120 uses the biometric information of the search target person, and thereby selects the candidate who has a possibility of being the search target person. For example, the selection unit 120 uses the biometric information, and thereby selects, from the candidates stored in the candidate storage unit 160, the candidate having a possibility of being the search target person. For example, the selection unit 120 computes a similarity degree between the biometric information of the search target person and the biometric information stored in the candidate storage unit 160, and selects the candidate associated with the biometric information whose similarity degree is equal to or higher than a reference value. Herein, the selection unit 120 selects a plurality of the candidates in one case, or selects only one candidate in another case, or selects no candidates in still another case.

Note that, when the search target person or the candidate is a missing person, the selection unit 120 may uses at least one of a time from the time when the biometric information of the missing person is generated to the current time and the information concerning an age of the missing person, and thereby change a reference for selecting the candidate. One example of this reference is the above-described reference value of a similarity degree. In this case, the selection unit 120 lowers the reference value as a time from the time when the biometric information of the missing person is generated to the current time becomes longer, because of a decline in reliability of the biometric information. The selection unit 120 lowers the reference value as an age of the missing person is smaller, because of a decline in reliability of the biometric information. Such a manner can reduce a possibility that selection leakage occurs in a selection result of the selection unit 120.

When the search target person or the candidate is a missing person, the selection unit 120 may use biometric information of a relative of the missing person, thereby modify the biometric information of the missing person, and select the candidate by using the modified biometric information. The reason for this is that the biometric information of the missing persons is highly likely to change with aging, but a tendency of this change is highly likely to be similar to a tendency of a change in biometric information of the relative. For example, the selection unit 120 changes the biometric information of the missing person in accordance with the tendency of the change in the biometric information of the relative, and selects the candidate by using the changed biometric information. Such a manner can reduce a possibility that selection leakage occurs in a selection result of the selection unit 120. Note that, one example of items of the biometric information to be subjected to such processing is face information.

The information generation unit 130 generates the possibility information by using the medical information of the search target person and the medical information of the candidate. A specific example of the possibility information is described above with reference to FIG. 2. For example, when the selection unit 120 selects a plurality of the candidates, the information generation unit 130 selects a person who is among a plurality of the candidates and who has a high possibility of being the search target person, and sets, as at least a part of the possibility information, information that indicates the person and whose example is a name and an address thereof.

Note that, there is a case where the medical information includes a plurality of items. In this case, weighting information such as weighting factors may be applied to these items. In this case, the information generation unit 130 computes a similarity degree for each item in a case of generating the possibility information, and uses the above-described weighting information at the time of integrating a plurality of these similarity degrees. For example, the information generation unit 130 may multiply a similarity degree for each item by the weighting factor associated with the item, add the multiplied values, and thereby compute a similarity degree that is actually used at the time of generating the possibility information. Such a manner improves accuracy of the possibility information generated by the information generation unit 130.

Note that, this weighting factor may change depending on, for example, a length of a time from the time when the search target person or the candidate goes missing. For example, the weighting factor may be lowered as time lapses, for the item that is highly likely to change as time lapses and whose example is the item concerning a chronic disease.

When the search target person or the candidate is a missing person, the information generation unit 130 may generate the possibility information by using at least one of: a time from the time when medical information of the missing person is generated to the current time; and information concerning an age of the missing person. In one example, the information generation unit 130 may select a used item in the medical information, depending on a time from the time when medical information of the missing person is generated to the current time. Such a manner improves accuracy of the possibility information generated by the information generation unit 130. For example, when a time from the time when medical information of the missing person is generated to the current time exceeds a reference, at the time of generating the possibility information, the information generation unit 130 does not use an item that is in the medical information and that is highly likely to change as time lapses. When the person went missing before becoming an adult, the information generation unit 130 does not use an item that is in the medical information and that is highly likely to change as time lapses. This is because reliability of the item in the medical information is presumed to be low.

When the search target person or the candidate is a missing person, the information generation unit 130 may modify at least a part of the medical information of the missing person by using medical information of a relative, such as a parent or a sibling, of the missing person. There is a possibility that medical information has a similar tendency between the person himself or herself and the relative. In one example, there is a possibility that a tendency of how a result of a blood test changes with aging is similar between the relatives. There is a possibility that a skeleton change occurring with growth or aging is also similar between the relatives. In view of the above, on the assumption that at least a part of items of the medical information of the missing person has changed in the tendency similar to that of the medical information of the relative, the information generation unit 130 modifies the item in accordance with this assumption, and generates the possibility information by using the modified item. Such a manner improves accuracy of the possibility information generated by the information generation unit 130.

Note that, the information generation unit 130 may further generate the possibility information by using the item before the item is modified. In this case, the information generation unit 130 generates at least two types of the possibility information.

The output unit 140 transmits, to the terminal 20, the possibility information generated by the information generation unit 130. The terminal 20 outputs this possibility information.

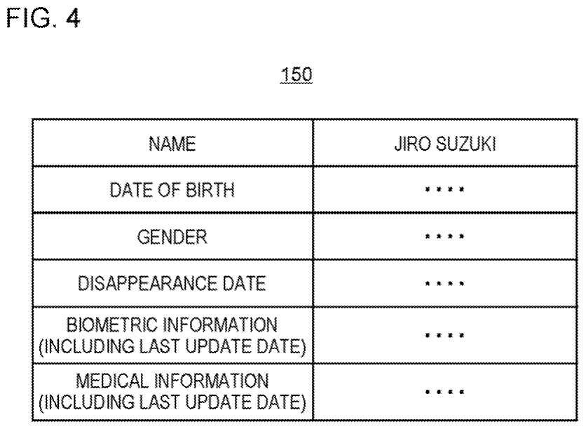

FIG. 4 is a diagram illustrating one example of information stored by the search target person storage unit 150. In the example illustrated in the present drawing, the search target person is a missing person. The search target person storage unit 150 stores a name, information with which an age can be determined (e.g., a date of birth), a gender, a disappearance date, the biometric information and the last update date thereof, and the medical information and the last update date thereof, for each search target person.

Note that, concerning at least one of relatives, such as parents and siblings, of the search target person, the search target person storage unit 150 may store information similar to that of the search target person. These pieces of information are also transmitted to the person determination apparatus 10 via the terminal 20 by the user, for example.

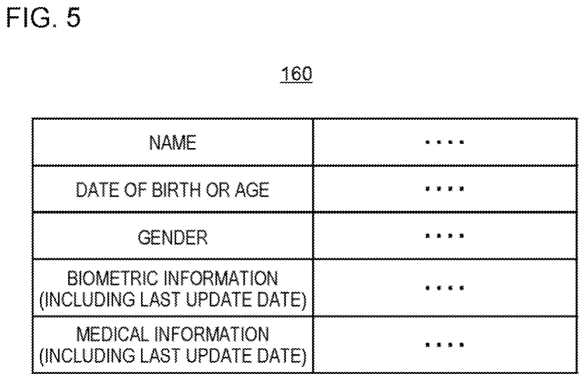

FIG. 5 is a diagram illustrating one example of information stored by the candidate storage unit 160. In the example illustrated in the present drawing, the candidate is an unidentified person. The candidate storage unit 160 stores a name, information with which an age can be determined (e.g., a date of birth, or an age), a gender, the biometric information and the last update date thereof, and the medical information and the last update date thereof, for each candidate. Note that, in some cases, the name and the information with which the age can be determined are provisional information.

Note that, when the search target person is an unidentified person, and the candidate is a missing person, the candidate storage unit 160 is used instead of the search target person storage unit 150, and the search target person storage unit 150 is used instead of the candidate storage unit 160.

The search target person storage unit 150 and the candidate storage unit 160 are used also when the search target person receives treatment after the search target person can be determined. For example, past medical information of the search target person is used as information of what is called a medical chart. When the medical information of his or her parent is included in the search target person storage unit 150, this medical information such as a medical history of the parent may be used. The information stored in the search target person storage unit 150 and the candidate storage unit 160 may be used at the time of notifying the search target person of recommendation information for maintaining health.

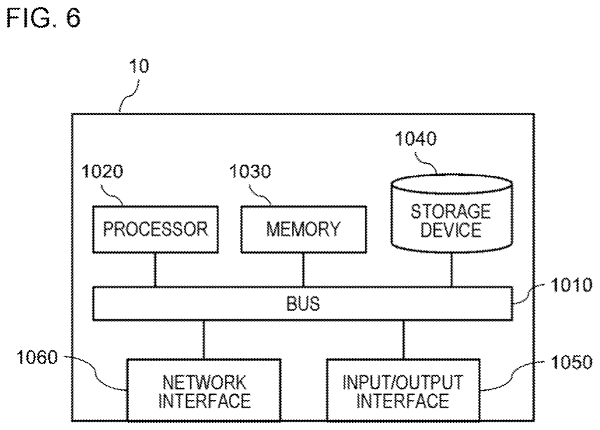

FIG. 6 is a diagram illustrating a hardware configuration example of the person determination apparatus 10. The person determination apparatus 10 includes a bus 1010, a processor 1020, a memory 1030, a storage device 1040, an input/output interface 1050, and a network interface 1060.

The bus 1010 is a data transmission path for allowing the processor 1020, the memory 1030, the storage device 1040, the input/output interface 1050, and the network interface 1060 to mutually transmit and receive data. However, a method of connecting the processor 1020 and the like to one another is not limited to bus connection.

The processor 1020 is a processor implemented by a central processing unit (CPU), a graphics processing unit (GPU), or the like.

The memory 1030 is a main storage device implemented by a random access memory (RAM) or the like.

The storage device 1040 is an auxiliary storage device implemented by a hard disk drive (HDD), a solid state drive (SSD), a removable medium such as a memory card, a read only memory (ROM), or the like, and includes a storage medium. The storage medium of the storage device 1040 stores a program module that implements each function (e.g., the acquisition unit 110, the selection unit 120, the information generation unit 130, and the output unit 140) of the person determination apparatus 10. The processor 1020 reads each of the program modules onto the memory 1030 and executes the read program module, and thereby, each function associated with the program module is implemented. In addition, the storage device 1040 also functions as the search target person storage unit 150 and the candidate storage unit 160.

The input/output interface 1050 is an interface for connecting the person determination apparatus 10 and various pieces of input/output equipment with each other.

The network interface 1060 is an interface for connecting the person determination apparatus 10 to a network. This network is a local area network (LAN) or a wide area network (WAN), for example. A method in which the network interface 1060 connects to the network may be a wireless connection, or may be a wired connection. The person determination apparatus 10 may communicate with the terminal 20 via the network interface 1060.

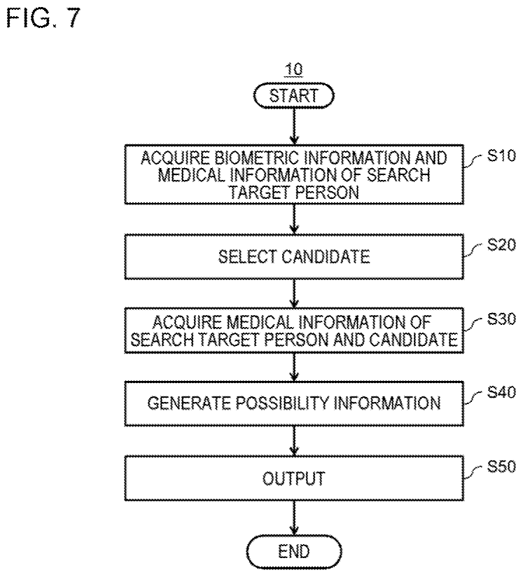

FIG. 7 is a flowchart illustrating a first example of processing performed by the person determination apparatus 10. First, the user inputs, to the terminal 20, information for determining the biometric information and the medical information of the search target person. This information may be the biometric information and the medical information themselves, or may be information whose example is a name and that is used for determination of the search target person stored in the search target person storage unit 150. The terminal 20 transmits the input information to the person determination apparatus 10.

The acquisition unit 110 of the person determination apparatus 10 acquires the biometric information of the search target person (step S10). In this processing, the acquisition unit 110 acquires the biometric information of the search target person from the terminal 20 in one case, or reads out the biometric information of the search target person from the search target person storage unit 150 by using the information acquired from the terminal 20 in another case.

Next, the selection unit 120 of the person determination apparatus 10 uses the biometric information acquired by the acquisition unit 110, and thereby selects, from among the candidates stored in the candidate storage unit 160, a person having a possibility of being a search target person (step S20). A specific example of the processing performed here is described above with reference to FIG. 3.

Next, the information generation unit 130 acquires the medical information of the search target person, and also acquires, from the candidate storage unit 160, the medical information of the candidate selected by the selection unit 120. In this processing, the information generation unit 130 acquires the medical information of the search target person from the terminal 20 in one case, or reads out the medical information of the search target person from the search target person storage unit 150 by using the information acquired from the terminal 20 in another case (step S30).

Next, the information generation unit 130 generates the possibility information. A specific example of the processing performed here is described above with reference to FIG. 3 (step S40). Then, the output unit 140 transmits the possibility information to the terminal 20 (step S50).

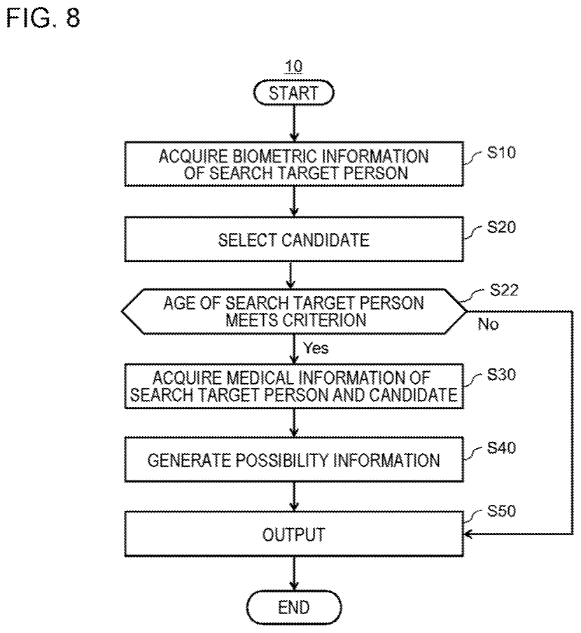

FIG. 8 is a flowchart illustrating an altered example of FIG. 7. The example illustrated in the present drawing is similar to the example illustrated in FIG. 7 except that the processing (steps S30 and S40) for generating the possibility information is performed by the information generation unit 130 when an age of the search target person meets the criterion (step S22: yes). Note that, when an age of the search target person does not meet the criterion (step S22: no), the processing for generating the possibility information is not performed. Then, the output unit 140 transmits, to the terminal 20, the information that concerns the candidate selected by the selection unit 120 and whose example is a face image (step S50).

Note that, the step S22 is performed since there is a possibility that reliability of the matching based on the medical information is low when the search target person is a small child. Note that, one example of the criterion for an age of the search target person is that an age of the search target person is equal to or over a reference value. This reference value is appropriately selected from ages before adulthood.

As described above, according to the present example embodiment, the person determination apparatus 10 includes the information generation unit 130 in addition to the selection unit 120. Thus, the person determination apparatus 10 can use the medical information in addition to the biometric information at the time of determining the search target person from among the candidates. Accordingly, using the person determination apparatus 10 enables the search target person to be determined from among the candidates, with high accuracy.

Although the example embodiment of the present invention is described above with reference to the drawings, these described matters are exemplifications of the present invention, and various configurations other than those described above can also be employed.

In addition, in a plurality of the flowcharts used in the above description, a plurality of the steps (pieces of processing) are described in order, but the execution order of the steps executed in each example embodiment is not limited to the described order. In each example embodiment, the order of the illustrated steps can be changed within a range in which inconvenience does not occur in the contents. The above-described each example embodiment can be combined within a range in which contradiction does not occur in the contents.

A part or all of the above-described example embodiment can also be described as in the following supplementary notes, but there is no limitation to the following.

1. A person determination apparatus including:
a selection unit that uses biometric information of a search target person, and thereby selects a candidate having a possibility of being the search target person; and
an information generation unit that uses medical information concerning a medical history of the search target person and the medical information of the candidate, and thereby generates possibility information being information concerning a possibility that the candidate is the search target person.
   2. The person determination apparatus according to the supplementary note 1, in which
the selection unit selects a plurality of the candidates, and
the information generation unit selects a person who is among the plurality of candidates and who has a high possibility of being the search target person, and sets, as at least a part of the possibility information, information indicating the selected person.
   3. The person determination apparatus according to the supplementary note 1 or 2, in which
the medical information includes at least one item of skeleton information, information concerning a chronic disease, information concerning a blood test, information concerning treatment received in a past, and information concerning a trace left in a body due to past treatment.
   4. The person determination apparatus according to the supplementary note 3, in which
the medical information includes a plurality of items, and
the information generation unit generates the possibility information by using weighting information set for each of the plurality of items.
   5. The person determination apparatus according to any one of the supplementary notes 1 to 4,
the information generation unit performs processing for generating the possibility information when an age of the search target person meets a criterion.
   6. The person determination apparatus according to any one of the supplementary notes 1 to 5, in which
the search target person or the candidate is a missing person, and
the information generation unit generates the possibility information by using at least one of a time from time when medical information of the missing person is generated to current time, and information concerning an age of the missing person.
   7. The person determination apparatus according to any one of the supplementary notes 1 to 6, in which
the search target person or the candidate is a missing person, and
the information generation unit uses the medical information of a relative of the missing person, and thereby modifies at least a part of the medical information of the missing person.
   8. The person determination apparatus according to the supplementary note 7, in which
the item that is in the medical information and that is modified includes skeleton information.
   9. The person determination apparatus according to any one of the supplementary notes 1 to 8, in which
the search target person or the candidate is a missing person, and
the selection unit uses at least one of a time from time when the biometric information of the missing person is generated to current time, and information concerning an age of the missing person, and thereby changes a reference for selecting the candidate.
   10. The person determination apparatus according to any one of the supplementary notes 1 to 9, in which
the search target person or the candidate is a missing person, and
the selection unit uses the biometric information of a relative of the missing person, thereby modifies biometric information of the missing person, and selects the candidate by using the modified biometric information.
   11. The person determination apparatus according to the supplementary note 10, in which
the item that is in the biometric information and that is modified includes face information.
   12. A person determination method including,
by a computer:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.
   13. The person determination method according to the supplementary note 12, including:
by the computer,
selecting a plurality of the candidates, and
selecting a person who is among the plurality of candidates and who has a high possibility of being the search target person, and setting, as at least a part of the possibility information, information indicating the selected person.
   14. The person determination method according to the supplementary note 12 or 13, in which
the medical information includes at least one item of skeleton information, information concerning a chronic disease, information concerning a blood test, information concerning treatment received in a past, and information concerning a trace left in a body due to past treatment.
   15. The person determination method according to the supplementary note 14, in which
the medical information includes a plurality of items, and
the method includes:
by the computer,
generating the possibility information by using weighting information set for each of the plurality of items.
   16. The person determination method according to any one of the supplementary notes 12 to 15, including:
by the computer,
performing processing for generating the possibility information when an age of the search target person meets a criterion.
   17. The person determination method according to any one of the supplementary notes 12 to 16, in which
the search target person or the candidate is a missing person, and
the method includes:
by the computer,
generating the possibility information by using at least one of a time from time when medical information of the missing person is generated to current time, and information concerning an age of the missing person.
   18. The person determination method according to any one of the supplementary notes 12 to 17, in which
the search target person or the candidate is a missing person, and
the method includes:
by the computer,
using the medical information of a relative of the missing person, and thereby modifying at least a part of the medical information of the missing person.
   19. The person determination method according to the supplementary note 18, in which
the item that is in the medical information and that is modified includes skeleton information.
   20. The person determination method according to any one of supplementary notes 12 to 19, in which
the search target person or the candidate is a missing person, and
the method includes:
by the computer,
using at least one of a time from time when the biometric information of the missing person is generated to current time, and information concerning an age of the missing person, and thereby changes a reference for selecting the candidate.
   21. The person determination method according to any one of the supplementary notes 12 to 20, in which
the search target person or the candidate is a missing person,
the method includes:
by the computer,
using the biometric information of a relative of the missing person, thereby modifying biometric information of the missing person, and selecting the candidate by using the modified biometric information.
   22. The person determination method according to the supplementary note 21, in which
the item that is in the biometric information and that is modified includes face information.
   23. A program causing a computer to include:
a selection unit that uses biometric information of a search target person, and thereby selects a candidate having a possibility of being the search target person; and
an information generation unit that uses medical information concerning a medical history of the search target person and the medical information of the candidate, and thereby generates possibility information being information concerning a possibility that the candidate is the search target person.
   24. The program according to the supplementary note 23, in which
the selection unit selects a plurality of the candidates, and
the information generation unit selects a person who is among the plurality of candidates and who has a high possibility of being the search target person, and sets, as at least a part of the possibility information, information indicating the selected person.
   25. The program according to the supplementary note 23 or 24, in which
the medical information includes at least one item of skeleton information, information concerning a chronic disease, information concerning a blood test, information concerning treatment received in a past, and information concerning a trace left in a body due to past treatment.
   26. The program according to the supplementary note 25, in which
the medical information includes a plurality of items, and
the information generation unit generates the possibility information by using weighting information set for each of the plurality of items.
   27. The program according to any one of the supplementary notes 23 to 26, in which
the information generation unit performs processing for generating the possibility information when an age of the search target person meets a criterion.
   28. The program according to any one of the supplementary notes 23 to 27, in which
the search target person or the candidate is a missing person, and
the information generation unit generates the possibility information by using at least one of a time from time when medical information of the missing person is generated to current time, and information concerning an age of the missing person.
   29. The program according to any one of the supplementary notes 23 to 28, in which
the search target person or the candidate is a missing person, and
the information generation unit uses the medical information of a relative of the missing person, and thereby modifies at least a part of the medical information of the missing person.
   30. The program according to the supplementary note 29, in which
the item that is in the medical information and that is modified includes skeleton information.
   31. The program according to any one of the supplementary notes 23 to 30, in which
the search target person or the candidate is a missing person, and
the selection unit uses at least one of a time from time when the biometric information of the missing person is generated to current time, and information concerning an age of the missing person, and thereby changes a reference for selecting the candidate.
   32. The program according to any one of the supplementary notes 23 to 31, in which
the search target person or the candidate is a missing person, and
the selection unit uses the biometric information of a relative of the missing person, thereby modifies biometric information of the missing person, and selects the candidate by using the modified biometric information.
   33. The program according to the supplementary note 32, in which
the item that is in the biometric information and that is modified includes face information.
   34. A storage medium that stores the program according to any one of the supplementary notes 23 to 33.

1. A person determination apparatus comprising:
at least one memory configured to store instructions; and
at least one processor configured to execute the instructions to perform operations comprising:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

2. The person determination apparatus according to claim 1, wherein
selecting the candidate comprises selecting a plurality of the candidates,
the operations further comprise selecting a person who is among the plurality of candidates and who has a high possibility of being the search target person, and
generating the possibility information comprises setting, as at least a part of the possibility information, information indicating the selected person.

3. The person determination apparatus according to claim 1, wherein
the medical information includes at least one item of skeleton information, information concerning a chronic disease, information concerning a blood test, information concerning treatment received in a past, and information concerning a trace left in a body due to past treatment.

4. The person determination apparatus according to claim 1, wherein
generating the possibility information comprises performing processing for generating the possibility information when an age of the search target person meets a criterion.

5. The person determination apparatus according to claim 1, wherein
the search target person or the candidate is a missing person, and
generating the possibility information comprises generating the possibility information by using at least one of a time from time when medical information of the missing person is generated to current time, and information concerning an age of the missing person.

6. The person determination apparatus according to claim 1, wherein
the search target person or the candidate is a missing person, and
the operations further comprise using the medical information of a relative of the missing person, and thereby modifying at least a part of the medical information of the missing person.

7. The person determination apparatus according to claim 1, wherein
the search target person or the candidate is a missing person, and
the operations further comprise using at least one of a time from time when the biometric information of the missing person is generated to current time, and information concerning an age of the missing person, and thereby changing a reference for selecting the candidate.

8. The person determination apparatus according to claim 1, wherein
the search target person or the candidate is a missing person,
the operations further comprise using the biometric information of a relative of the missing person, thereby modifying biometric information of the missing person, and
selecting the candidate comprises selecting the candidate by using the modified biometric information.

9. A person determination method comprising,
by a computer:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

10. The person determination method according to claim 9, wherein
selecting the candidate comprises selecting a plurality of the candidates,
the person determination method further comprise, by the computer, selecting a person who is among the plurality of candidates and who has a high possibility of being the search target person, and
generating the possibility information comprises setting, as at least a part of the possibility information, information indicating the selected person.

11. The person determination method according to claim 9, wherein
the medical information includes at least one item of skeleton information, information concerning a chronic disease, information concerning a blood test, information concerning treatment received in a past, and information concerning a trace left in a body due to past treatment.

12. The person determination method according to claim 9, wherein
generating the possibility information comprises performing processing for generating the possibility information when an age of the search target person meets a criterion.

13. The person determination method according to claim 9, wherein
the search target person or the candidate is a missing person, and
generating the possibility information comprises generating the possibility information by using at least one of a time from time when medical information of the missing person is generated to current time, and information concerning an age of the missing person.

14. The person determination method according to claim 9, wherein
the search target person or the candidate is a missing person, and
the person determination method further comprise, by the computer, using the medical information of a relative of the missing person, and thereby modifying at least a part of the medical information of the missing person.

15. A non-transitory storage medium storing a program causing a computer to execute a person determination method, the person determination method comprising:
using biometric information of a search target person, and thereby selecting a candidate having a possibility of being the search target person; and
using medical information concerning a medical history of the search target person and the medical information of the candidate, and thereby generating possibility information being information concerning a possibility that the candidate is the search target person.

16. The non-transitory storage medium according to claim 15, wherein
selecting the candidate comprises selecting a plurality of the candidates,
the person determination method further comprise selecting a person who is among the plurality of candidates and who has a high possibility of being the search target person, and
generating the possibility information comprises setting, as at least a part of the possibility information, information indicating the selected person.

17. The non-transitory storage medium according to claim 15, wherein
the medical information includes at least one item of skeleton information, information concerning a chronic disease, information concerning a blood test, information concerning treatment received in a past, and information concerning a trace left in a body due to past treatment.

18. The non-transitory storage medium according to claim 15, wherein
generating the possibility information comprises performing processing for generating the possibility information when an age of the search target person meets a criterion.

19. The non-transitory storage medium according to claim 15, wherein
the search target person or the candidate is a missing person, and
generating the possibility information comprises generating the possibility information by using at least one of a time from time when medical information of the missing person is generated to current time, and information concerning an age of the missing person.

20. The non-transitory storage medium according to claim 15, wherein
the search target person or the candidate is a missing person, and
the person determination method further comprise using the medical information of a relative of the missing person, and thereby modifying at least a part of the medical information of the missing person.
